(19) United States
(12) Patent Application Publication
(10) Pub. No.: US 2022/0361791 A1
(43) Pub. Date: Nov. 17, 2022

(54) URODYNAMIC MEASUREMENT APPARATUS FOR ADJUSTING THE HEIGHT OF THE SENSOR

(71) Applicant: SapienMed Co., Ltd, Seoul (KR)

(72) Inventor: Seung June OH, Seoul (KR)

(73) Assignee: SapienMed Co., Ltd, Seoul (KR)

(21) Appl. No.: 17/569,515

(22) Filed: Jan. 6, 2022

Related U.S. Application Data
(60) Provisional application No. 63/188,414, filed on May 13, 2021.

(51) Int. Cl. A61B 5/20; A61B 5/03; A61B 5/00

(52) U.S. Cl. CPC A61B 5/205; A61B 5/036; A61B 5/6852; A61B 5/4561; A61B 2562/0247

(57) ABSTRACT

The present disclosure provides a urodynamic measurement apparatus that includes a urethral catheter that may be inserted inside a patient's bladder, a first sensor that may be spaced apart from the patient, and connected to the urethral catheter to measure an inner pressure of the bladder, a rectal catheter that may be inserted inside the patient's rectum, a second sensor that may be spaced apart from the patient, but adjacent to the first sensor, and connected to the rectal catheter to measure an abdominal pressure, and a first position adjuster that adjusts the height of the first sensor and the second sensor according to the patient's posture.

### 1. FIELD

The present disclosure relates to an apparatus for adjusting positions of a bladder pressure sensor and an abdominal pressure sensor of a urodynamic measurement apparatus.

### 2. BACKGROUND

Storing and emptying urine properly is achieved through integration of functions of the urinary bladder and urethral sphincter and the nervous system that controls these organs. Urodynamic study is an in-depth examination for diagnosing functional abnormalities of the urinary bladder and urethral sphincter. Urodynamic study is a collective term for tests made up of several detailed items such as uroflowmetry, filling cystometry, pressure-flow study, urethral pressure profile, and urethral sphincter electromyography. These tests are conducted selectively for patients with dysuria and urine incontinence.

Medical staff can gain detailed information necessary to accurately identify the characteristics of lower urinary tract dysfunction through urodynamic study. Based on the results of urodynamic study, medical staff can clearly distinguish between various types of bladder and urethral dysfunctions that can be expressed clinically even in a single disease entity.

The most important role of urodynamic study is to provide detailed information necessary for the medical staff to determine the prognosis of patients with specific urinary tract dysfunctions and establish a treatment plan. Based on the findings of the lower urinary tract dysfunction in the urodynamic study, medical staff can provide the best treatment method applicable to the patient.

Urodynamic study is the single most important test in determining the prognosis and treatment of the lower urinary tract dysfunction. Therefore, it is necessary for the medical staff to accurately interpret the results of a patient's urodynamic study.

FIG. 1A is a graph showing the intravesical pressure (Pves), abdominal pressure (Pabd), detrusor pressure (Pdet) and urethral sphincter electromyography (EMG) activity measured during the filling cystometry of a patient in a supine position.

Referring to FIG. 1A, urodynamic study includes several detailed measurements that identify various indicators related to the function of storing and emptying urine. Urodynamics study allows the medical staff to interpret the results of the intravesical pressure (Pves) 11, abdominal pressure (Pabd) 12, detrusor pressure (Pdet) 13, and urethral sphincter activity 14 information of the patient measured through a urodynamic measurement apparatus as aforementioned, making it possible to diagnose the patient's lower urinary tract dysfunction.

Here, the detrusor pressure (Pdet) 13 cannot be measured directly by the urodynamic measurement apparatus, and thus it is displayed as a virtual value obtained by subtracting the abdominal pressure (Pabd) 12 from the intravesical pressure (Pves) 11 in real time through computer calculation.

Prior to performing the urodynamic study correctly, basic preparation processes such as calibration and zeroing are required. Urodynamic study indicators such as pressure and urinary flow that are currently being used are values that are measured as electrical signals and then reinterpreted in real time as actual values. Therefore, calibration must be done between those actual values and electrical signal measurement values on a regular basis if not every time.

Meanwhile, zeroing must be done not only every time the patient has the urodynamic study, but also when the patient has each detailed test item during the urodynamic study. In the case of the urodynamic study, there is not just one pressure transducer, that is, there is the intravesical pressure and abdominal pressure, and thus the electrical signal values of each of the pressure transducers have different references for conversion.

Especially, in the urodynamic study, detrusor pressure involves calculating the difference between the intravesical pressure and the abdominal pressure in real time, but if these two values have different references, the measured values might represent completely different values, and thus it is very important to equalize the reference heights and adjust the zero point (zeroing). Therefore, for each patient, right before conducting the urodynamic study, the patient is placed in a supine position and the reference heights of these transducers are zeroed, and then the study starts. With the patient in the supine position, the filling cystometry is conducted, and then for the pressure-flow study, generally the patient is placed in a standing position or a sitting position. Therefore, every time the patient changes their posture for each detailed test, zeroing is conducted again.

To zero the pressures, first, the position of the pressure transducer is set to the reference height at the atmospheric pressure, and then zeroed, and then the pressure is measured. The International Continence Society (ICS) has set the upper edge of the symphysis pubis to be the reference height, and this is called the ICS reference level.

When performing the detailed tests of the urodynamic study, the tests may be performed after changing the testing posture of the patient to a standing position, sitting position, or obliquely lying position (semi-Fowler's position). Each time the position is changed, the position of the upper edge of the symphysis pubis is changed, and thus the reference height of the pressure transducer must be manually adjusted to the position of the patient's upper edge of the symphysis pubis each time. Meanwhile, during the urodynamic study, the patient may change their posture to a supine position, sitting position, or standing position, and during this process, there occurs a difference of height between the pressure transducer and the catheter end located in the patient's bladder or rectum, which will affect the measurement values of the intravesical pressure (Pves) 11 and the abdominal pressure (Pabd) 12.

However, a conventional urodynamic study measurement apparatus has a problem that the difference in the height of the catheter end occurring due to the change of the patient's posture is reflected in the measurement value as it is.

FIG. 1B is a graph showing the intravesical pressure, abdominal pressure, detrusor pressure and urethral sphincter activity measured during filling cystometry when the patient changes their posture from the supine position to standing position.

Further referring to FIG. 1B, one can see that at the time point (t1) when the patient changes their position from the supine position to standing position, the intravesical pressure (Pves) 11 and abdominal pressure (Pabd) 12 sharply increase.

That is, in a conventional urodynamic study measurement apparatus, there is a problem that the measurement values of the intravesical pressure (Pves) and abdominal pressure (Pabd) are affected according to the changes in the position of the catheter end, and this problem may become a cause for the medical staff to misdiagnose.

### SUMMARY

Therefore, a purpose of the present disclosure is to resolve the aforementioned problem of prior art, that is, to provide a urodynamic measurement apparatus for adjusting the sensor position, capable of adjusting the position of a sensor and a catheter end according to a patient's posture.

A urodynamic measurement apparatus according to an embodiment of the present disclosure includes a urethral catheter that may be inserted inside a patient's bladder; a first sensor that may be spaced apart from the patient, and connected to the urethral catheter to measure an inner pressure of the bladder; a rectal catheter that may be inserted inside the patient's rectum; a second sensor that may be spaced apart from the patient, but adjacent to the first sensor, and connected to the rectal catheter to measure an abdominal pressure; and a first position adjuster that adjusts the height of the first sensor and the second sensor according to the patient's posture.

The first position adjuster may include a transfer rail; a mounting frame that is coupled in a transferrable manner in an up and down direction on the transfer rail, and where the first sensor and the second sensor may be mounted; and a signal transmitter and receiver that measures a distance between the mounting frame and the patient's upper edge of the symphysis pubis according to the patient's posture, and the mounting frame may be transferred in the up and down direction on the transfer rail such that the distance between the mounting frame and the patient's upper edge of the symphysis pubis becomes the shortest.

The urodynamic measurement apparatus may further include a second position adjuster that adjusts each of the height of the first sensor and the second sensor according to the position of the tip of the urethral catheter and the tip of the rectal catheter.

The second position adjuster may include a first height adjuster that may be installed on the mounting frame, and adjust the height of the first sensor; and a second height adjuster that may be installed on the mounting frame such that it is adjacent to the first height adjuster, and adjust the height of the second sensor.

The urodynamic measurement apparatus may further include a posture detector that detects the patient's posture, and the first position adjuster may adjust each of the height of the first sensor and the second sensor according to the patient's posture detected by the posture detector.

The urodynamic measurement apparatus for adjusting the sensor position of the present disclosure may provide medical staff with data about the intravesical pressure, abdominal pressure and detrusor pressure that are not affected by a patient's posture change, and therefore, the medical staff can interpret the patient's test results more accurately without any test errors relating to zeroing.

### BRIEF DESCRIPTION OF THE DRAWINGS

FIG. 1A is a graph showing the intravesical pressure (Pves), abdominal pressure (Pabd), detrusor pressure (Pdet) and urethral sphincter activity measured during filling cystometry of a patient in a supine position;

FIG. 1B is a graph showing the intravesical pressure, abdominal pressure, detrusor pressure and urethral sphincter activity measured during filling cystometry when the patient changes their posture from the supine position to standing position;

FIG. 2 is a view for describing a part of the urodynamic measurement apparatus for adjusting the sensor position according to an embodiment of the present disclosure;

FIG. 3 is a concept view of the urodynamic measurement apparatus for adjusting the sensor position according to an embodiment of the present disclosure mounted onto a patient in a supine position;

FIG. 4 is a concept view of the urodynamic measurement apparatus for adjusting the sensor position according to an embodiment of the present disclosure mounted onto a patient in a standing position;

FIG. 5 is a view for describing a first position adjuster of the urodynamic measurement apparatus of FIG. 3;

FIG. 6 is a view describing an end of a urethral catheter and an end of a rectal catheter of the urodynamic measurement apparatus of FIG. 3 inserted into the bladder and the rectum, respectively, with the patient in a supine position;

FIG. 7 is a view describing an end of a urethral catheter and an end of a rectal catheter of the urodynamic measurement apparatus of FIG. 4 inserted into the bladder and the rectum, respectively, with the patient in a standing position;

FIG. 8 is a block diagram of the urodynamic measurement apparatus of FIG. 3; and

FIG. 9 is a view for describing a second position adjuster of the urodynamic measurement apparatus of FIG. 3.

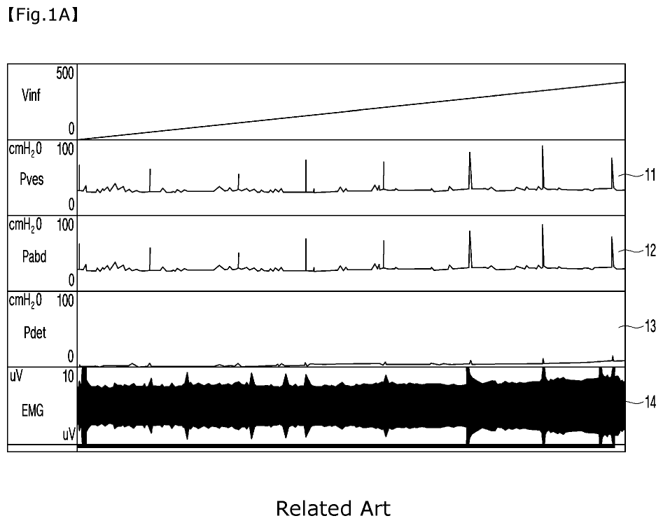

### DETAILED DESCRIPTION

Hereinbelow, some embodiments of the present disclosure will be described in detail through the exemplary drawings. In adding reference numerals to components of each drawing, it should be noted that even if the components are displayed on different drawings, like reference numerals are used for like components as much as possible.

Further, in describing the embodiments of the present disclosure, if it is determined that a specific description of a related well-known configuration or a function interrupts the understanding of the embodiments of the present disclosure, detailed description thereof will be omitted.

Further, in describing the components of the present disclosure, terms such as a first, a second, A, B, (a), (b) and the like may be used. Such terms are merely used to distinguish those components from other components, but such terms do not limit the nature, sequence or order of the corresponding components.

Hereinbelow, referring to the accompanying drawings, the urodynamic measurement apparatus for adjusting the sensor position according to an embodiment of the present disclosure will be described in detail.

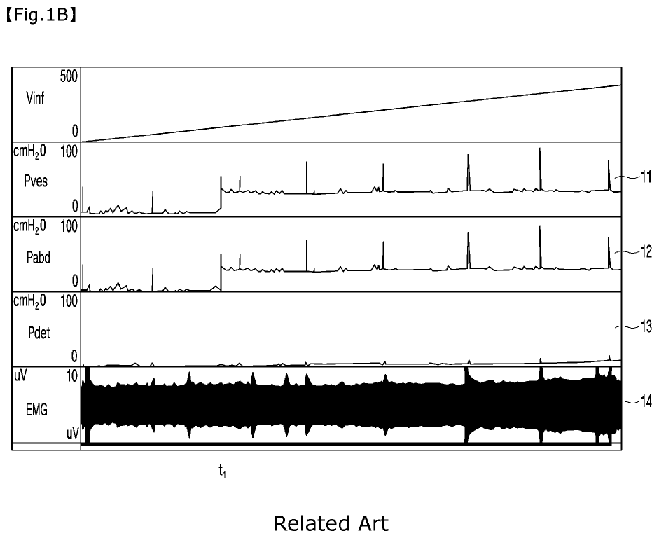

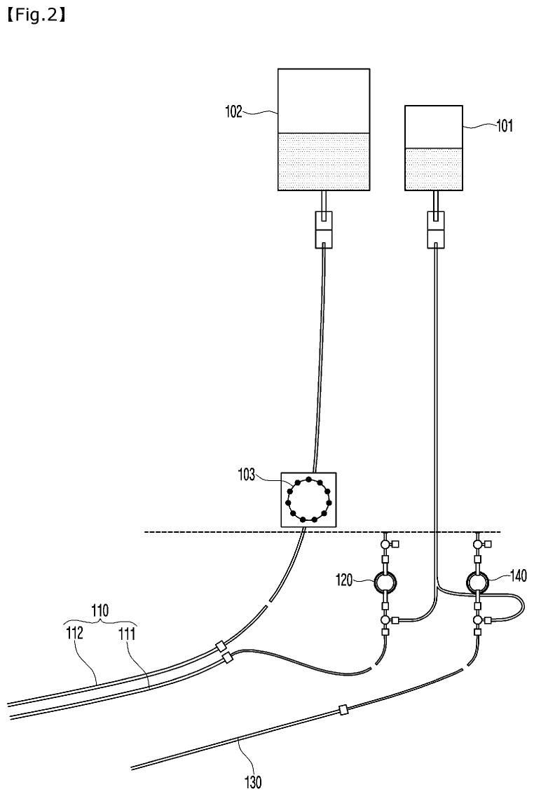

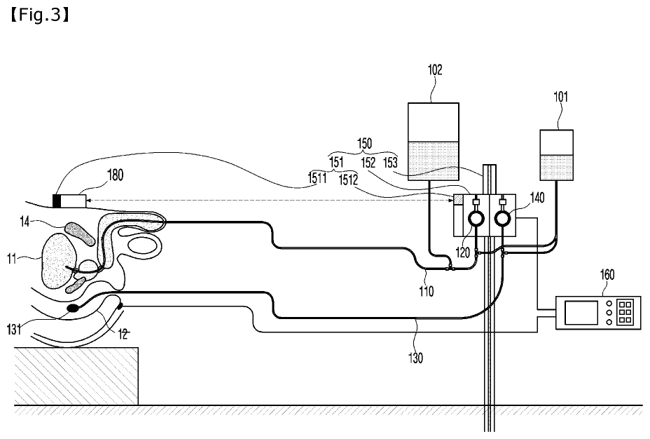

FIG. 2 is a view for describing a part of the urodynamic measurement apparatus for adjusting the sensor position according to an embodiment of the present disclosure, FIG. 3 is a concept view of the urodynamic measurement apparatus for adjusting the sensor position according to an embodiment of the present disclosure mounted onto a patient in a supine position, and FIG. 4 is a concept view of the urodynamic measurement apparatus for adjusting the sensor position according to an embodiment of the present disclosure mounted onto a patient in a standing position.

Referring to FIGS. 2 to 4, the urodynamic measurement apparatus for adjusting the sensor position according to an embodiment of the present disclosure 100 (hereinafter referred to as “urodynamic measurement apparatus”) includes a urethral catheter 110 that may be inserted inside a patient's bladder 11, a rectal catheter 130 that may be inserted inside the patient's rectum, a first pressure transducer 120 (hereinafter referred to as “first sensor”) that may be connected to the urethral catheter 110 to measure an inner pressure of the patient's bladder 11, a second pressure transducer 140 (hereinafter referred to as “second sensor”) that may be connected to the rectal catheter 130 to measure the patient's abdominal pressure, a first pocket 101 that may be connected to each of the first sensor 120 and the second sensor 140, to provide fluid such as physiological saline to the first sensor 120, the second sensor 140, the urethral catheter 110 and the rectal catheter 130, and a second pocket 102 that may be connected to the urethral catheter 110 to provide fluid such as physiological saline to the urethral catheter 110.

The urethral catheter 110 may include a first flow path 111 that may be connected to the first sensor 120, and a second flow path 112 that may be connected to the second pocket. The first flow path 111 and the second flow path 112 of the urethral catheter 110 may be formed inside one tube independently.

A pump 103 is provided between the second pocket 102 and the second flow path 112, and the pump 103 may introduce the fluid of the second pocket 102 to the patient's bladder 11 through the second flow path 112.

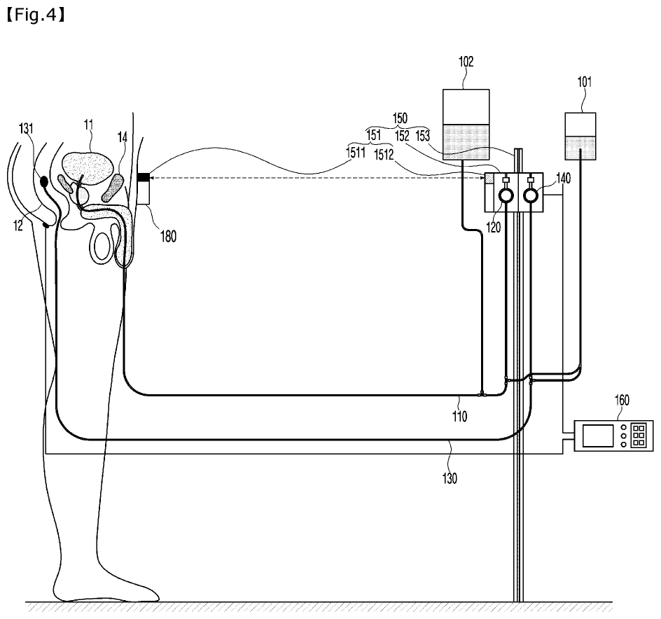

FIG. 5 is a view for describing a first position adjuster of the urodynamic measurement apparatus of FIG. 3.

Referring to FIG. 5 further, the urodynamic measurement apparatus 100 further includes a first position adjuster 150 and a controller 160.

The urethral catheter 110 is inserted into the patient's bladder 11 through the patient's urethra such that a tip portion of the urethral catheter 110 is positioned in the bladder 11.

When the fluid in the first pocket 101 is injected into the first sensor 120 and the first flow path 111, from an end portion of the first flow path 111, the fluid is discharged to the patient's bladder 111, and here the first sensor 120 may measure the inner pressure of the patient's bladder 11 by the method of measuring the fluid pressure of the first flow path 111.

The first sensor 120 may measure the inner pressure of the patient's bladder 11 during a filling phase where the fluid such as physiological saline is supplied to the patient's bladder 11 through the second flow path 112, and during a voiding phase where the fluid such as physiological saline filled in the patient's bladder 11 is discharged through the urethra.

The rectal catheter 130 is inserted into the rectum 12 through the patient's anus such that a tip portion of the rectal catheter 130 is positioned in the patient's rectum 12.

At the tip portion of the rectal catheter 130, a rectal balloon 131 may be provided, and the rectal balloon 131 may be filled with fluid such as physiological saline.

When the fluid in the first pocket 101 is injected into the second sensor 140 and the rectal catheter 130, the fluid is introduced into the rectal balloon 131 positioned at the tip of the rectal catheter 130.

Here, the second sensor 140 may measure the patient's abdominal pressure in the method of measuring the fluid pressure of the rectal catheter 130.

The urodynamic measurement apparatus 100 may further include an electromyography electrode 165.

The electromyography electrode 165 is attached near the patient's anus to measure the urethral sphincter activity, which may be used to determine whether the detrusor contraction is synergic or dyssynergic with the urethral sphincter activity.

The controller 160 may be connected with the first sensor 120, the second sensor 140 and the electromyography electrode 165, and based on signals received from the first sensor 120, the second sensor 140 and the electromyography electrode 165, the controller 160 may display the intravesical pressure (Pves) 11, the abdominal pressure (Pabd) 12, the detrusor pressure (Pdet) 13, and the urethral sphincter activity 14 through a display.

The first sensor 120 and the second sensor 140 are mounted onto the first position adjuster 150.

The first position adjuster 150 may adjust the height of the first sensor 120 and the second sensor 140 according to the height of the upper edge of the symphysis pubis (ICS reference level), which is the reference height.

That is, the first position adjuster 150 may change the height of the first sensor 120 and the second sensor 140 by following the height of the patient's upper edge of the symphysis pubis which changes according to the patient's posture.

For this purpose, the first position adjuster 150 includes a signal transmitter and receiver 151, a mounting frame 152, and a transfer rail 153.

The transfer rail 153 is disposed vertically to the ground, and on the mounting frame 152, each of the first sensor 120 and the second sensor 140 are installed, and the mounting frame 152 may move in an up and down direction on the transfer rail 153.

Specifically, the mounting frame 152 may move in an up and down direction on the transfer rail 153 by the driving force of an actuator such as a motor.

The signal transmitter and receiver 151 may measure a distance between the patient's upper edge of the symphysis pubis 14 and the mounting frame 152.

The signal transmitter and receiver 151 includes a receiver 1511 and a transmitter 1512.

One of the receiver 1511 and the transmitter 1512 is installed on the patient's upper edge of the symphysis pubis 14, and the other one is installed on the mounting frame 152, so as to measure the distance between the patient's upper edge of the symphysis pubis 14 and the mounting frame 152.

For example, the receiver 1511 may be installed on the patient's upper edge of the symphysis pubis 14 while the transmitter 1512 is installed on the mounting frame 152, to measure the distance between the patient's upper edge of the symphysis pubis 14 and the mounting frame 152.

Using the time in which the signal transmitted from the transmitter 1512 is received in the receiver 1511, the distance between the transmitter 1512 and the receiver 1511 may be measured.

The signal that the transmitter 1512 and the receiver 1511 transmits and receives may be any signal such as ultrasound, vision, infrared, and visible light, as long as it is capable of measuring distance.

The controller 160 may adjust the position of the mounting frame 152 on the transfer rail 153 in an up and down direction such that the distance between the patient's upper edge of the symphysis pubis 14 and the mounting frame 152 is maintained to the minimum distance based on the distance between the transmitter 1512 and the receiver 1511 measured in the signal transmitter and receiver 151.

For example, if the height of the first sensor 120 and the second sensor 140 is higher than that of the patient's upper edge of the symphysis pubis 14, a difference of height will occur between the transmitter 1512 and the receiver 1511, and the distance L2 between the transmitter 1512 and the receiver 1511 will become greater than the reference distance L1. In this case, the controller 160 may transfer the transfer rail 153 downwards on the mounting frame 152, such that the distance between the transmitter 1512 and the receiver 1511 can be the same as the reference distance L1.

Further, even when the height of the first sensor 120 and the second sensor 140 is lower than that of the patient's upper edge of the symphysis pubis 14, there will occur a height difference between the transmitter 1512 and the receiver 1511, and the distance L3 between the transmitter 1512 and the receiver 1511 will become greater than the reference distance L1. In this case, the controller 160 may transfer the transfer rail 153 upwards on the mounting frame 152, such that the distance L1 between the transmitter 1512 and the receiver 1511 can be the same as the reference distance L1.

Here, the reference distance L1 refers to the distance between the transmitter 1512 and the receiver 1511 when the height of the patient's upper edge of the symphysis pubis 14 is the same as the height of the first sensor 120 and the second sensor 140, and the controller 160 may transfer the transfer rail 153 on the mounting frame 152, to find a point where the distance between the transmitter 1512 and the receiver 1511 becomes minimum, and determine it as the reference distance L1.

Therefore, the present disclosure is able to track the height of the patient's upper edge of the symphysis pubis 14 according to the patient's posture, and control such that the height of the first sensor 120 and the second sensor 140 becomes the same as the height of the patient's upper edge of the symphysis pubis 14.

Specifically, when the patient changes their posture such as the supine position, sitting position, and standing position, the position of the receiver 1511 being installed on the patient's upper edge of the symphysis pubis 14 will change, in which case, the transmitter 1512 will be moved in an up and down direction by the mounting frame 152, to track the height of the patient's upper edge of the symphysis pubis 14 in search for the shortest distance between the transmitter 1512 and the receiver 1511, and thus the height of the first sensor 120 and the second sensor 140 will be following the height of the patient's upper edge of the symphysis pubis 14.

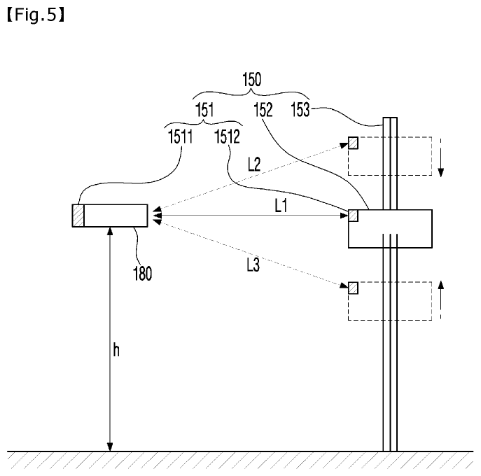

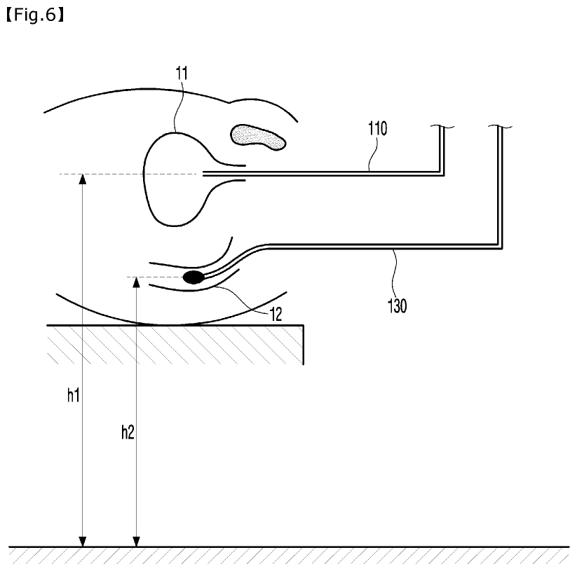

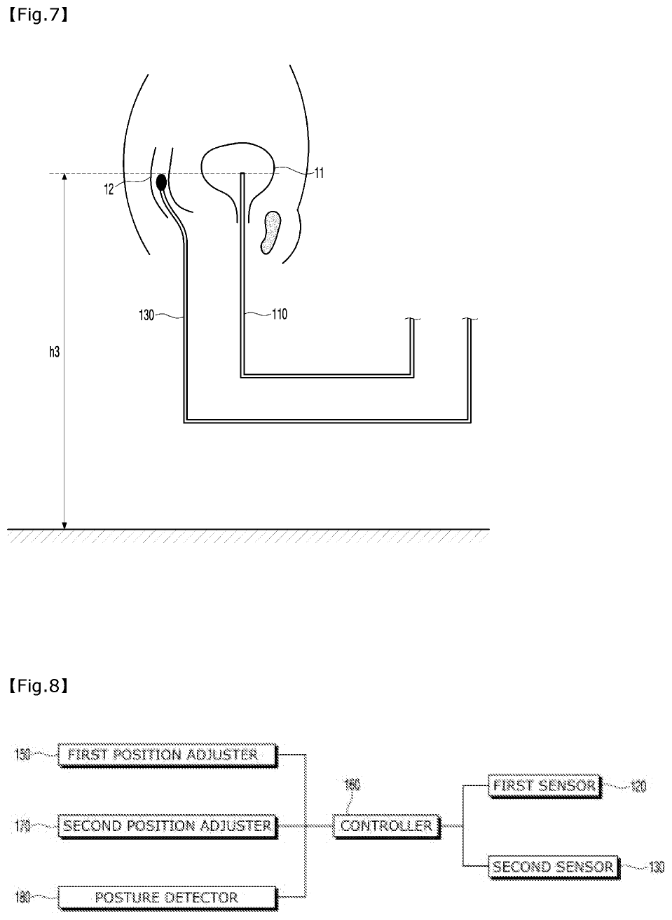

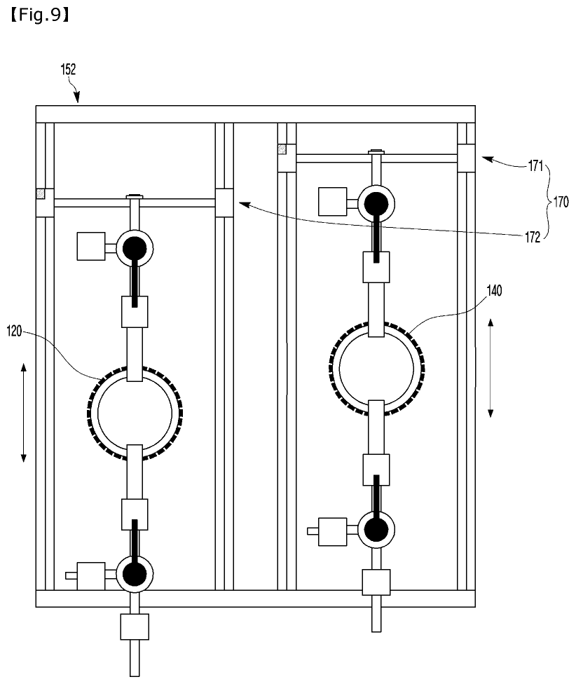

FIG. 6 is a view describing an end of a urethral catheter and an end of a rectal catheter of the urodynamic measurement apparatus of FIG. 3 inserted into the bladder and the rectum, respectively, with the patient in a supine position, FIG. 7 is a view describing an end of a urethral catheter and an end of a rectal catheter of the urodynamic measurement apparatus of FIG. 4 inserted into the bladder and the rectum, respectively, with the patient in a standing position, FIG. 8 is a block diagram of the urodynamic measurement apparatus of FIG. 3, and FIG. 9 is a view for describing a second position adjuster of the urodynamic measurement apparatus of FIG. 3.

Referring to FIGS. 6 to 9 further, the urodynamic measurement apparatus 100 may further include a second position adjuster 170.

The second position adjuster 170 may adjust the height of the first sensor 120 and the second sensor 140 according to the position of the tip of the urethral catheter 110 and the tip of the rectal catheter 130.

The second position adjuster 170 may be installed on the mounting frame 152.

The second position adjuster 170 may include a first height adjuster 171 and a second height adjuster 172.

The first height adjuster 171 and the second height adjuster 172 may each have an actuator such as a motor, so as to be slided in an up and down direction on the mounting frame.

The first sensor 120 may be installed on the first height adjuster 171, and the first sensor 120 may be transferred in an up and down direction on the mounting frame 152 by the first height adjuster 171.

The second sensor 140 is installed on the second height adjuster 172.

The second height adjuster 172 may be adjacent to the first height adjuster 171, and may move in an up and down direction on the mounting frame 152. The second sensor 140 may be transferred in an up and down direction on the mounting frame 152 by the second height adjuster 172.

That is, the second position adjuster 170 may transfer each of the height of the first sensor 120 and the height of the second sensor 140, individually.

The second position adjuster 170 may be characterized to adjust each of the height of the first sensor 120 and the second sensor 140 according to the patient's posture.

For this purpose, the urodynamic measurement apparatus 100 may further include a posture detector 180.

The posture detector 180 detects the patient's posture such as the supine position, sitting position and standing position.

The posture detector 180 may consist of an acceleration sensor and a gyro sensor.

Based on the information detected in the posture detector 180, the controller 160 may control the second position adjuster 170 to adjust the height of the first sensor 120 and the second sensor 140.

Specifically, according to the change of height of the tip of the urethral catheter 110 and the tip of the rectal catheter 130 occurring due to the difference of height of the patient's bladder 11 and the rectum 12, as the patient changes their posture, the controller 160 may control the second position adjuster 170 to adjust each of the height of the first sensor 120 and the second sensor 140.

Hereinabove, even if all components constituting an embodiment of the present disclosure are described as being combined into one or operating in combination, the present disclosure is not necessarily limited to this embodiment. That is, as long as it is within the scope of the object of the present disclosure, one or more of all those components may be selectively combined and operated.

Further, all terms, including technical or scientific terms disclosed hereinabove, have the same meaning as commonly understood by one of ordinary skill in the art, unless otherwise defined. Terms generally used, such as terms defined in the dictionary, should be interpreted as being consistent with the meaning of the context of the related technology, and are not interpreted as ideal or excessively formal meanings unless explicitly defined in the present disclosure.

Further, the above description is merely illustrative of the technical idea of the present disclosure, and various modifications and variations may be made by those of ordinary skill in the technical field to which the present disclosure pertains, without departing from the essential characteristics of the present disclosure. Therefore, the embodiments disclosed in the present disclosure are not intended to limit the technical idea of the present disclosure, but to describe it, and the scope of the technical idea of the present disclosure is not limited by these embodiments. The scope of protection of the present disclosure should be interpreted by the following claims, and all technical ideas within the scope equivalent thereto should be construed as being included in the scope of the present disclosure.

What is claimed is:

1. A urodynamic measurement apparatus comprising:
a urethral catheter that may be inserted inside a patient's bladder;
a first sensor that may be spaced apart from the patient, and connected to the urethral catheter to measure an inner pressure of the bladder;
a rectal catheter that may be inserted inside the patient's rectum;
a second sensor that may be spaced apart from the patient, but adjacent to the first sensor, and connected to the rectal catheter to measure an abdominal pressure; and
a first position adjuster that adjusts the height of the first sensor and the second sensor according to the patient's posture.

2. The urodynamic measurement apparatus according to claim 1,
wherein the first position adjuster comprises a transfer rail;
a mounting frame that is coupled in a transferrable manner in an up and down direction on the transfer rail, and where the first sensor and the second sensor may be mounted; and
a signal transmitter and receiver that measures a distance between the mounting frame and the patient's upper edge of the symphysis pubis according to the patient's posture,
wherein the mounting frame is transferred in the up and down direction on the transfer rail such that the distance between the mounting frame and the patient's upper edge of the symphysis pubis becomes the shortest.

3. The urodynamic measurement apparatus according to claim 2,
further comprising a second position adjuster that adjusts each of the height of the first sensor and the second sensor according to the position of the tip of the urethral catheter and the tip of the rectal catheter.

4. The urodynamic measurement apparatus according to claim 3,
wherein the second position adjuster comprises a first height adjuster that may be installed on the mounting frame, and adjust the height of the first sensor; and
a second height adjuster that may be installed on the mounting frame such that it is adjacent to the first height adjuster, and adjust the height of the second sensor.

5. The urodynamic measurement apparatus according to claim 3,
further comprising a posture detector that detects the patient's posture,
wherein the first position adjuster adjusts each of the height of the first sensor and the second sensor according to the patient's posture detected by the posture detector.
